(19) United States
(12) Patent Application Publication
(10) Pub. No.: US 2012/0021076 A1
(43) Pub. Date: Jan. 26, 2012

(54) METHOD FOR TONING THE FACE SKIN

(76) Inventors: Mark Constantine, Dorset (GB); Margaret Joan Constantine, Dorset (GB); Helen Elizabeth Ambrosen, Dorset (GB)

(21) Appl. No.: 12/995,837

(22) PCT Filed: Jun. 2, 2009
(86) PCT No.: PCT/GB2009/001369

§ 371 Date: Oct. 7, 2011

(30) Foreign Application Priority Data
Jun. 3, 2008 (GB) 0810116.4

(51) Int. Cl. A61K 8/97; A61Q 19/00

(52) U.S. Cl. 424/745; 424/769

(57) ABSTRACT

The present invention provides a method of toning the face comprising the steps of (a) dissolving in water a toner concentrate in the form of a solid comprising (i) citric acid; (ii) sodium bicarbonate; (iii) one or more essential oils; to provide a toner, and (b) applying the toner to the face.

### FIELD OF THE INVENTION

The present invention relates to a method of toning the face, to a toner used in the method, a concentrate for preparing the toner and to a kit comprising the concentrate and instructions to use in the method.

### BACKGROUND TO THE INVENTION

Toners are a category of cosmetic products, which are used to refresh the skin and the senses. They are also used to remove traces of make-up and any cleanser left on the skin after it has been cleaned.

Conventionally toners are mixtures of components which predominantly contain water. Examples of components present in toners are as follows. Alcohol, such as ethanol may be added to de-grease the skin. Fragrance is also often present and is an important ingredient of toners. Fragrances are typically added when mixed with the alcohol content. Alternatively it may be solublised with a suitable cosmetic ingredient such as PEG 60 almond glycerides and/or PEG 60 caprylic/capric glycerides. These types of material function to dissolve oil based fragrances in water based toner products.

Toners contain cosmetic preservatives. The large water content of toners requires preservatives to prevent growth of spoilage or potentially harmful micro-organisms.

After formulation toners are typically packaged in bottles. The bottle are usually of plastic or glass construction and protect the toner from the environment and to keep in a suitable condition during use.

The care of some skin types, particularly those with acne prone skin, takes some effort. As long as the skin is cared for carefully and consistently good effects can be achieved and maintained. However, prior art other products have been found not to ‘engage’ the user. This has resulted in low usage rates of products and resultant deterioration in skin condition.

The present invention seeks to give an effective but pleasurable way of completing the cleansing process. This encouraged consistency with cleansing, leading to improved skin care.

### SUMMARY OF THE INVENTION

In a first aspect, there is provided a method of toning the face comprising the steps of

(a) dissolving in water a toner concentrate in the form of a solid comprising
(i) citric acid
(ii) sodium bicarbonate
(iii) one or more essential oils
to provide a toner
   (b) applying the toner to the face.

In a second aspect, there is provided a kit comprising

(a) a facial toner concentrate in the form of a solid comprising
(i) citric acid
(ii) sodium bicarbonate
(iii) one or more essential oils
   (b) instructions to dissolve the toner concentrate in water to provide a toner and to apply the toner to the face.

In a third aspect, there is provided a facial toner obtained or obtainable by dissolving in water a facial toner concentrate in the form of a solid comprising

(i) citric acid
(ii) sodium bicarbonate
(iii) one or more essential oils

In a fourth aspect, there is provided a facial toner concentrate in the form of a solid comprising

(i) citric acid
(ii) sodium bicarbonate
(iii) one or more essential oils

For ease of reference, these and further aspects of the present invention are now discussed under appropriate section headings. However, the teachings under each section are not necessarily limited to each particular section.

### ADVANTAGES

The toner of the present invention and for use in the present invention is freshly made when required for use. It is only during dissolution that the active constituents of the toner product are combined with large amounts of water. Because of this, the spoilage problems of prior art toners are not encountered. Thus the toner concentrate, and therefore the toner of the present invention need not contain cosmetic preservatives or solublisers. This is advantageous because these types of cosmetic materials can cause responses of irritation or allergy on the skin.

The present toner produced from a toner concentrate is kinder to the environment as it does not need to be stored in a container and avoids the need to transport a material with is primarily composed of water.

The solid nature of the concentrate is further advantageous for users who are travelling with the product. Not only is the weight and volume of the product reduced, in scenarios where volume of liquid carried is restricted, such as during airline flights, the present product allows for the user to still carry the product

### DETAILED DESCRIPTION

### Method

As discussed herein, in one aspect the present invention provides a method of toning the face comprising the steps of

(a) dissolving in water a toner concentrate in the form of a solid comprising
(i) citric acid
(ii) sodium bicarbonate
(iii) one or more essential oils
to provide a toner
   (b) applying the toner to the face.

Preferably, the method of the present invention comprises the additional step of placing the face in proximity to the water during dissolution of the toner concentrate in the water.

In a further preferred aspect, the toner concentrate is dissolved in water at a temperature of at least 40° C., such as at a temperature of at least 50° C., such as a temperature of at least 60° C. or such as at a temperature of at least 70° C., or such as at a temperature of at least 80° C., or such as at a temperature of from 40 to 95° C.

If the above steps are combined, namely the additional step of placing the face in proximity to the water during dissolution of the toner concentrate in the water is performed and the toner concentrate is dissolved in water at a temperatures above (such as at least 40° C.) the user may ‘steam’ their face prior to cleaning with the toner. Steaming the face with warm steam from hot water is a traditional form of cleaning. It has been found to be very effective for all skin types, though particularly for problem skin which is troubled with spots or a texture where the pores are open. This attracts and traps dirt and sebum.

Thus in use, the concentrate may be dropped in to a sink or bowl of hot steamy water. The face in then placed in proximity to the dissolving concentrate. Preferably a towel may placed over the head for a minute or two. The steam then envelops the skin to warm and soften it. The water produced is used on cotton wool to remove excess cleanser before moisturising and to refresh the skin and the senses. The face, neck and shoulders are the main areas of the body for which the toner would be used. Refreshing beneath the arms in the armpits would also be another use, especially if travelling.

The effect of the toners in the present invention is heightened because of the way they are used. Dissolved in hot water, the steam generated warms the skin allowing ingredients to pass more easily in to the deeper layers. Aromatic essential oils dissolve in the steam. Their action improves the texture and condition of the skin. When the water is then used to wipe the face and neck, the water-soluble extracts and vitamins are applied to the skin, soothing and calming or degreasing the complexion.

The manner in which facial toners are typically used is application of the toner to the face and to areas surrounding the face, such as the neck area. When facial toner is used it is typically not applied to other parts of the body either at the same time as application to the face or separately to application to the face and surrounding areas. Thus in a preferred aspect of the present method, step (b) comprises applying the toner to the face, neck or both and not applying the toner to other parts of the body. Thus in a preferred aspect of the present kit, (b) comprises instructions to dissolve the toner concentrate in water to provide a toner and to apply the toner to the face, neck or both and not to applying the toner to other parts of the body. In a preferred aspect of the present method, step (b) comprises applying the toner to the face, neck or both and not bathing in the toner. In a preferred aspect of the present kit, (b) comprises instructions to dissolve the toner concentrate in water to provide a toner and to apply the toner to the face, neck or both and not to bathe in the toner.

Thus, in summary, the present invention provides a solid material, such as a small solid tablet, which effervesces in water. The tablet releases active, effective ingredients and fragrance in the hot water it is dissolved in. Once the tablet has dissolved, the water is used to clean the face of any residual make-up or cleanser. This, for example, may be achieved by dipping cotton wool in to the water where the tablet has dissolved and then wiping the face and optionally neck with the cotton wool pad. The toner product is made up by the user by combination with water, for example in a sink or bowl.

### Kit

As discussed herein, in one aspect the present invention provides a kit comprising

(a) a facial toner concentrate in the form of a solid comprising
(i) citric acid
(ii) sodium bicarbonate
(iii) one or more essential oils
   (b) instructions to dissolve the toner concentrate in water to provide a toner and to apply the toner to the face.

The kit may comprises instructions to perform any of the steps or preferred aspects of the method described herein. For example the kit preferably contains instructions to perform the additional step of placing the face in proximity to the water during dissolution of the toner concentrate in the water. The kit may also preferably contain instructions to dissolve the toner concentrate in water at a temperature of at least 40° C.

### Toner

As discussed herein, in one aspect the present invention provides a facial toner obtained or obtainable by dissolving in water a facial toner concentrate in the form of a solid comprising

(i) citric acid
(ii) sodium bicarbonate
(iii) one or more essential oils

### Concentrate

As discussed herein, in one aspect the present invention provides a facial toner concentrate in the form of a solid comprising

(i) citric acid
(ii) sodium bicarbonate
(iii) one or more essential oils

The solid toner will typically be provided in the form a tablet. The tablet will typically have a volume of 1 to 20 cm³. For example the tablet will have a volume of 2 to 10 cm³, or 2 to 6 cm³, or 1 to 5 cm³, or 2 to 4 cm³. In addition or in one alternative, the tablet will typically have a diameter of up to 3 cm, or no less than 1 cm, or no less than 1 cm or from 1 to 4 cm or from 1 to 3 cm or from 1.5 to 3 cm.

In a preferred aspect the facial toner concentrate (or the other formed therefrom) further comprising one or more vitamins. Preferably the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the toner concentrate of a material, such as a natural material, that has a high vitamin content.

The one or more essential oils present in the facial toner concentrate (or the other formed therefrom) are preferably selected from tea tree oil and lavender oil. Other essential oils may be present such as rose oil.

The essential oils will be selected based on the fragrance desired, skin type to be treated and other effects desired based on the well known properties of essential oils. The addition of essential oils can when taken in to the nose are known to alter mood. For example, essential oils are known to create effects of drowsiness or stimulating the senses. Many, well documented effects, can be achieved by the use of essential oils. When the present concentrate is dissolved in water of a suitable temperature (see teaching herein) the steam generated by the water will enhance these effects.

It will be appreciated by one skilled in the art that many suitable active ingredients can be incorporated in to the formula. As discussed herein, vitamins, particularly B, C and E are very beneficial for the skin. Vitamin rich ingredients such as Wheatgerm oil can also be used to deliver vitamins on to the skin. Fruit and herb extracts and juices, vegetable oils and essential oils are all compatible with the formula. Colours, both naturally derived and synthetic can be used to colour the toner concentrate leading to the water in which they are dissolved being coloured as a result.

The ingredients in the present invention do not require cosmetic preservatives, solublisers or alcohols, such as ethanol. The use of cosmetic preservatives can increase the potential to irritate the skin. The use of alcohols can cause the skin to become dry. The ingredients in the present invention do not require cosmetic preservatives, as there is no water content. Equally, fragrances do not need to solublised and therefore solublisers can be avoided.

The present invention provides a facial toner concentrate in the form of a solid comprising

(i) citric acid
(ii) sodium bicarbonate
(iii) one or more essential oils

The following provides preferred amounts of each of the components. Each of these ranges may be taken alone or combined with one or more other component ranges to provided a preferred aspect of the invention

### Citric Acid: 25 to 37 wt %

### Sodium Bicarbonate: 62 to 80 wt %

### Fruit or Herb Extract or Fruit Juice: 0.01 to 1 wt %

Vitamins—Oil based form or powder: 0.01 to 1 wt %
Fragrance: 0.05 to 2.0 wt % (as a source of essential oils)

### Example

The invention will now be described with reference to the following non-limiting example.

A toner concentrate having the following composition was prepared.

The formula is as follows

| | Wt % |
|---|---|
| Sodium Bicarbonate | 73.25 |
| Ti tree oil* (an essential oil and fragrance) | 0.1 |
| Wheatgerm oil (as a vitamin source) | 0.05 |
| Aloe Vera extract | 0.05 |
| Citric Acid | 26.55 |
| TOTAL | 100 |

*also known as tea tree oil

The components of the composition were mixed together and the mixture was compressed to provide a toner concentrate in the form of a tablet.

The concentrate was dissolved in tap water at a temperature of approximately 50° C. The toner was then applied to the face of the tester using cotton wool soaked with toner. The tester found that the skin was well cleaner, as evidenced from the dirt on the cotton wool.

Alternative formulations are made by substituting the aloe vera extract with lemon juice in one embodiment and with lemon infusion in another embodiment.

An alternative formulation is made by substituting the ti tree oil with lavender essential oil.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A method of toning the face comprising the steps of
(a) dissolving in water a toner concentrate in the form of a solid comprising
(i) citric acid
(ii) sodium bicarbonate
(iii) one or more essential oils
to provide a toner
(b) applying the toner to the face.

2. A method according to claim 1 wherein comprising the additional step of placing the face in proximity to the water during dissolution of the toner concentrate in the water.

3. A method according to claim 1 wherein the toner concentrate is dissolved in water at a temperature of at least 40° C.

4. A method according to claim 1 wherein the facial toner concentrate further comprising one or more vitamins.

5. A method according to claim 4 wherein the vitamins are selected from vitamin C, vitamin E and mixtures thereof.

6. A method according to claim 1 wherein the one or more essential oils are selected from tea tree oil and lavender oil.

7. A method according to claim 1 wherein step (b) comprises applying the toner to the face and optionally the neck and not applying the toner to other parts of the body.

8. A kit comprising
(a) a facial toner concentrate in the form of a solid comprising
(i) citric acid
(ii) sodium bicarbonate
(iii) one or more essential oils
(b) instructions to dissolve the toner concentrate in water to provide a toner and to apply the toner to the face.

9. A kit according to claim 8 with instructions to perform the additional step of placing the face in proximity to the water during dissolution of the toner concentrate in the water.

10. A kit according to claim 8 with instructions to dissolve the toner concentrate in water at a temperature of at least 40° C.

11. A kit according to claim 8 wherein the facial toner concentrate further comprises one or more vitamins.

12. A kit according to claim 11 wherein the vitamins are selected from vitamin C, vitamin E and mixtures thereof.

13. A kit according to claim 8 wherein the one or more essential oils are selected from tea tree oil and lavender oil.

14. A kit according to claim 8 wherein (b) comprises instructions to dissolve the toner concentrate in water to provide a toner and to apply the toner to the face and optionally the neck and not to applying the toner to other parts of the body.

15. A facial toner obtained or obtainable by dissolving in water a facial toner concentrate in the form of a solid comprising
(i) citric acid
(ii) sodium bicarbonate
(iii) one or more essential oils

16. A facial toner according to claim 15 wherein the facial toner concentrate further comprising one or more vitamins.

17. A facial toner according to claim 16 wherein the vitamins are selected from vitamin C, vitamin E and mixtures thereof.

18. A facial toner according to claim 15 wherein the one or more essential oils are selected from tea tree oil and lavender oil.

19. A facial toner concentrate in the form of a solid comprising
(i) citric acid
(ii) sodium bicarbonate
(iii) one or more essential oils

20. A facial toner concentrate according to claim 19 further comprising one or more vitamins.

21. A facial toner concentrate according to claim 20 wherein the vitamins are selected from vitamin C, vitamin E and mixtures thereof.

22. A facial toner concentrate according to claim 19 wherein the one or more essential oils are selected from tea tree oil and lavender oil.

23. (canceled)

24. (canceled)

25. (canceled)

26. (canceled)
